(19) United States
(12) Patent Application Publication
(10) Pub. No.: US 2014/0240832 A1
(43) Pub. Date: Aug. 28, 2014

(54) MICRODRAPE

(71) Applicants: FUJI SYSTEMS CORPORATION, Tokyo (JP); MITAKA KOHKI CO., LTD., Tokyo (JP)

(72) Inventors: Katsushige NAKAMURA, TOKYO (JP); Katsuyuki NAKAMURA, TOKYO (JP); Hiroki SUGAIZUMI, TOKYO (JP)

(73) Assignees: FUJI SYSTEMS CORPORATION, Tokyo (JP); MITAKA KOHKI CO., LTD., Tokyo (JP)

(21) Appl. No.: 14/189,820

(22) Filed: Feb. 25, 2014

(30) Foreign Application Priority Data
Feb. 25, 2013 (JP) 2013-34876

(51) Int. Cl. A61B 19/08

(52) U.S. Cl. CPC A61B 19/081
USPC 359/510

(57) ABSTRACT

A microdrape 10 covers a surgical microscope system 60 including a surgical microscope 61 and a pair of binoculars 62 independently supported by an arm structure 64. The microdrape 10 has an opening end 6 and a closed end opposite to the opening end 6. The microdrape 10 includes: a cylindrical bag shaped main body 10a having the opening end 6; a first cylindrical bag 1 connected to the main body, 10a wherein the first cylindrical bag 1 has a hood for covering a lens tube of an objective lens 61a of the surgical microscope 61 and projecting portions 9a, 9b for covering lens tubes of eyeglasses 61b of the surgical microscope 61; and a second cylindrical bag 2 connected to the main body 10a, where the second cylindrical bag 2 has projecting portions 9c, 9d for covering lens tubes of eyeglasses 62a of the binoculars 62. A slit 5 is formed between the first cylindrical bag 1 and the second cylindrical bag 2. The microdrape can cover the surgical microscope 61, the binoculars 62 and the arm structure 64 without interfering with movement of the arm structure 64.

This application claims the benefit of Japanese Patent Application No. 2013-34876, filed Feb. 25, 2013, which is hereby incorporated by reference in its entirety.

### FIELD OF INVENTION

The present invention relates to a disposable drape for microscopes, namely a microdrape, which covers a surgical microscope and binoculars for assistant supported by an independent supporting arm structure.

### RELATED BACKGROUND ARTS

In neurosurgery and in ophthalmic surgery, an operation is performed with great care while enlarging a diseased area by employing a surgical microscope suspended from a universally movable arm structure. Since the surgical microscope cannot be sterilized, the surgical microscope is covered with a sterilized covering cloth called a microdrape so as to keep the surgical microscope clean during the operation.

For example, Japanese laid open patent No. 04-92656 discloses a microdrape used for the microscope, which is capable of firmly sticking to a lens tube and an arm of the microscope. Hereinafter, the disclosed microdrape is explained as referring to FIG. 7.

FIG. 7(a) is a plan view of a drape for a surgical microscope (hereinafter referred as “microdrape”) and FIG. 7(b) is a side view showing the microscope covered with the microdrape. A microdrape 81 shown in FIG. 7(a) is formed as a bag made of a quite thin and flexible transparent sheet. Reference numeral 82 is a connecting hem, where a front sheet and a rear sheet are connected together. Reference numeral 83 is an opening end of the microdrape. Reference numeral 84 is projecting portions where lens tubes of eyeglasses of the microscope are arranged. Reference numeral 85 is projecting portions where auxiliary instruments are arranged, and the instruments are attached to the main body of the microscope such as a side view mirror, TV camera and the like. Reference numerals 86 and 87 are perforation lines for easy removal of tip ends of the projecting portions 84 for the eyeglasses and tip ends of projecting portions 85 for auxiliary instruments, when the tip ends are pulled. Reference numeral 88 is a flexible cap for an objective lens of the microscope. Further, reference numeral 89 is a transparent plastic plate detachably inserted in the cap 88. Reference numeral 90 is an outlet to be connected to an aspirator, which will be explained below. Reference numerals 91, 92 and 93 are strings so as to stick the opening end 83 and the projecting portions 84 and 85 closely to the arm, lens tubes of the eyeglasses and the auxiliary instruments of the microscope, respectively.

Hereinafter, steps for attaching the drape 81 to the surgical microscope are explained as referring to FIG. 7(b). In the drawing, reference numeral 94 is a base of the surgical microscope. Reference numeral 95 is an arm horizontally rotatably attached to the base 94. Reference numeral 96 is a vertically movable and horizontally rotatable arm structure attached to the arm 95. Reference numeral 97 is a lens tube supported by the arm structure 96.

The lens tube 97, arm structure 96 and the arm 95 are put into the drape 81 via the opening end 83 in this order. After the respective lens tubes of eyeglasses are put into projecting portions 84 and knotted by the strings 92, the tip ends 84′ of the projecting portions 84 are pulled and separated apart from the portions 84. The objective lens is inserted in the lens cap 88. The arm 95 is knotted by the string 91 so as to close the opening end 83. In this way the surgical microscope structure is quarantined in a sterilized state. The projecting portions 85 for auxiliary instruments are folded and fitted to the microdrape by tapes or the like. If the auxiliary instruments are attached to the surgical microscope, projecting portions 85 are treated in the same way as the projecting portions 84 for the lens tubes. Then, an aspirator 99 is connected to an air outlet 90 via a suction pipe 98, so that air in the drape 81 can be exhausted by the aspirator 99.

And Japanese patent No. 3626142 discloses a microdrape which can be easily and completely fitted to a surgical microscope equipped with a grip formed in a bar shape.

### DISCLOSURE OF THE INVENTION

Recently surgical microscope systems equipped with one or more binoculars for assistant(s) as shown in FIGS. 8(a) and 8(b) have been prevailing. The assistants may move the binoculars independently from the surgical microscope for observing diseased areas.

The above-mentioned surgical microscope system comprises a camera for picking up images from a surgical microscope 61 via a branched optical path as electronic data. The electronic image data are displayed on display panel(s) of binoculars 62 so as to be observed by the assistant(s). FIG. 8(a) shows the system comprising a pair of binoculars 62 and FIG. 8(b) shows the system comprising two pairs of binoculars. In these systems, the binoculars 62 are not moved along with movements of the surgical microscope. Thus, a doctor D1 and assistants D2, (D3) can manipulate the surgical microscope 61 and the binoculars 62 independently.

However, the above-described conventional microdrape 81 cannot keep up with movements of the binoculars 62 independently supported by a holding arm 64 as shown in FIGS. 9(a) and 9(b), so that the movements of the holding arm 64 are restricted. Further, in some cases a lens tube of the surgical microscope is damaged by the tightened microdrape.

Embodiments of the present invention solve the problems mentioned above, and provide a microdrape used for a surgical microscope system comprising binoculars for assistants supported by a supporting arm structure independent from the surgical microscope. The microdrape can cover the surgical microscope, the binoculars for assistants and the independent arm structure for supporting the binoculars without interfering with movement of the independent supporting arm structure.

In order to solve the above-mentioned problems, the microdrapes in embodiments are characterized by (1) to (4).
(1) A microdrape for covering a surgical microscope system comprising a surgical microscope and one or more pair(s) of binoculars independently supported by one or more arm structure(s), wherein the microdrape has an opening end and a closed end opposite to the opening end, the microdrape comprising: a cylindrical bag shaped main body having the opening end; a first cylindrical bag connected to the main body, wherein the first cylindrical bag has a hood for covering a lens tube of an objective lens of the surgical microscope and projecting portions for covering lens tubes of eyeglasses of the surgical microscope; and one or more second cylindrical bag(s) connected to the main body, wherein the second cylindrical bag(s) has projecting portions for covering lens tubes of eyeglasses of the one or more pairs of binoculars, wherein: one or more slit(s) are formed between the first cylindrical bag and the second cylindrical bag(s).
(2) The microdrape according to (1), wherein: the surgical microscope has a plurality of eyeglasses, and the first cylindrical bag has a plurality of projecting portions for covering lens tubes of the plurality of eyeglasses of the surgical microscope.
(3) The microdrape according to (1) or (2), wherein: perforation lines are formed on tip ends of the projecting portions for covering the lens tubes of the eyeglasses of the surgical microscope and on tip ends of the projecting portions for covering the lens tubes of eyeglasses of the binoculars, and rubber rings are arranged near the perforation lines.
(4) The microdrape according to one of (1)-(3), wherein: a predetermined number of binding tapes are arranged on the cylindrical bag shaped main body a predetermined space apart.

The present invention can provide a microdrape used for surgical microscope systems comprising binoculars for assistants supported by a supporting arm structure independent from the surgical microscope. The microdrape can cover the surgical microscope, the binoculars for assistants and the independent supporting arm structure without interfering with movements of the independent supporting arm structure.

### BRIEF DESCRIPTION OF DRAWINGS

FIG. 1 shows an example of the surgical microscope system, (a) is a side view and (b) is a perspective view.

FIG. 2 shows a first embodiment of the microdrape. (a) is a plan view of the microdrape, (b) is a partially enlarged view of area A in FIGS. 2(a) and (c) is a side view showing a surgical microscope system covered with the microdrape shown in FIG. 2(a).

FIG. 3 shows a second embodiment of the microdrape. (a) is a plan view of the microdrape and (b) is a partial perspective view of a surgical microscope system to be covered with the microdrape shown in (a).

FIG. 4 shows a third embodiment of the microdrape. (a) is a plan view of the microdrape and (b) is a partial perspective view of a surgical microscope system to be covered with the microdrape shown in (a).

FIG. 5 shows a fourth embodiment of the microdrape. (a) is a plan view of the microdrape and (b) is a partial perspective view of a surgical microscope system to be covered with the microdrape shown in (a).

FIG. 6 shows a fifth embodiment of the microdrape. (a) is a plan view of the microdrape and (b) is a partial perspective view of a surgical microscope system to be covered with the microdrape shown in (a).

FIG. 7 shows a conventional microdrape. (a) is a plan view of the microdrape and (b) a side view showing the surgical microscope system covered with the microdrape shown in (a).

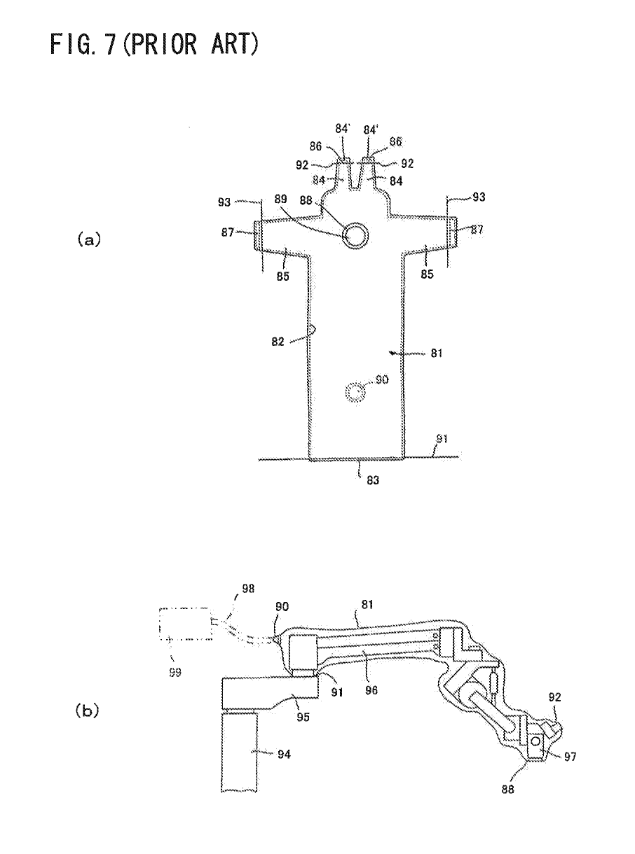

FIG. 8 shows surgical microscope systems equipped with (a) a pair of binoculars and (b) two pairs of binoculars for the assistant(s).

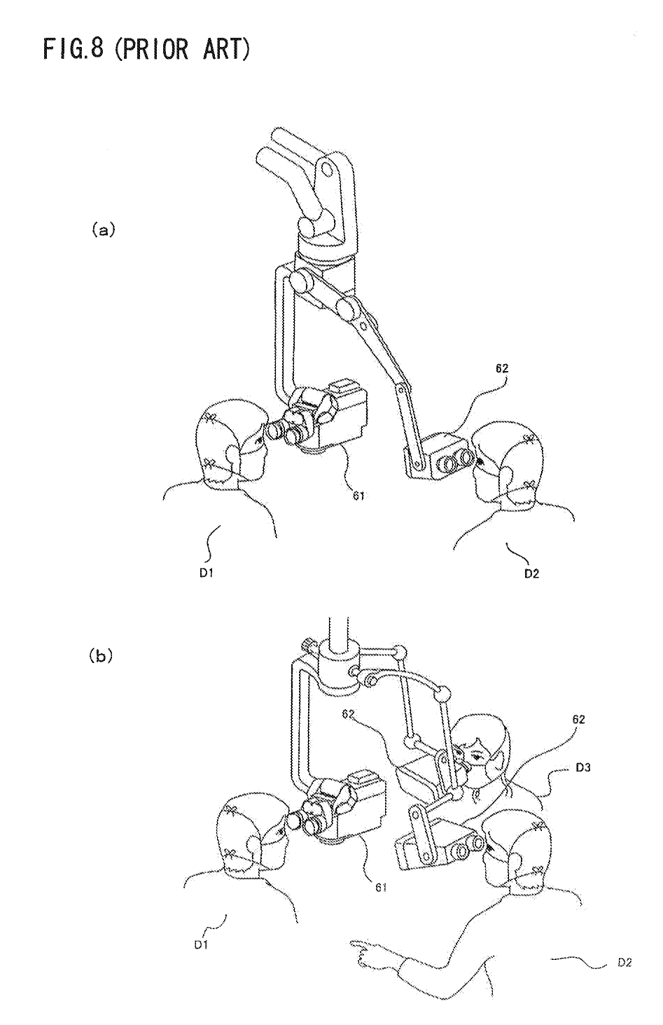

FIG. 9 shows (a) a moving area of a pair of binoculars in FIG. 8 and (b) a status of the binoculars covered with the conventional microdrape.

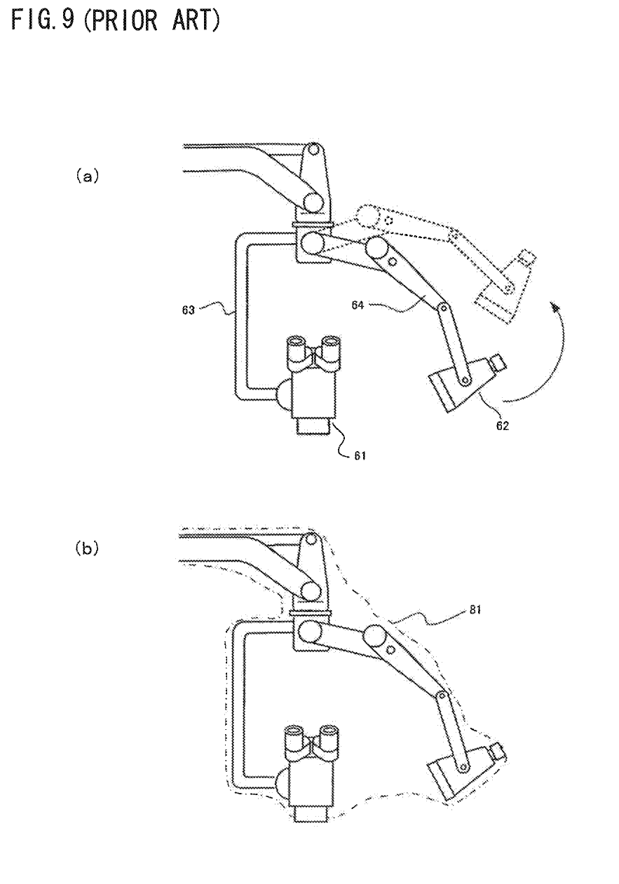

### EMBODIMENTS OF THE PRESENT INVENTION

Hereinafter, embodiments of the present invention are explained as referring to drawings.

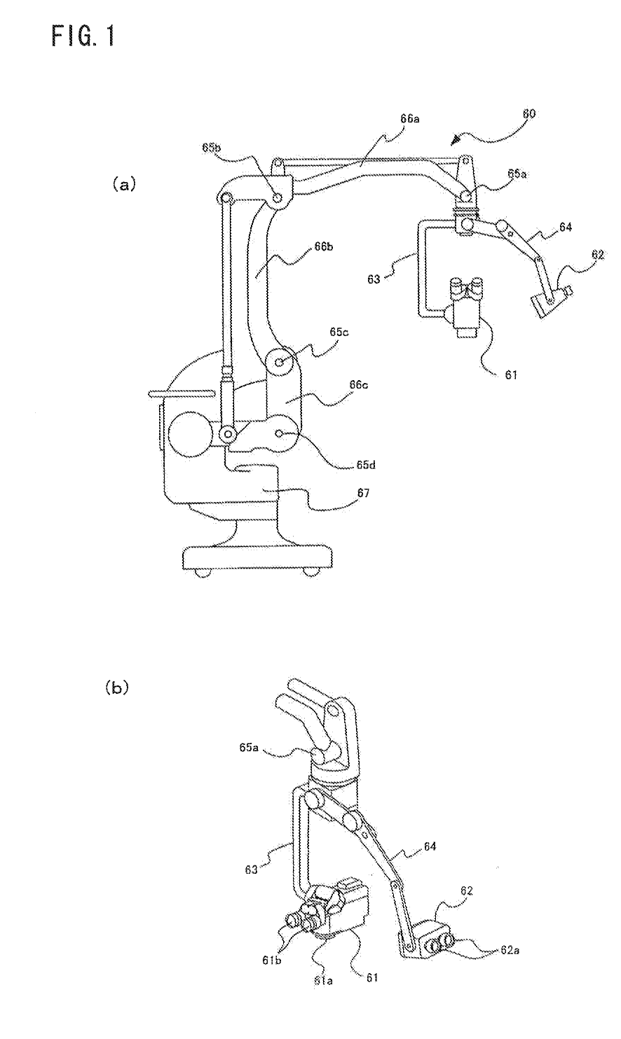

FIG. 1(a) is a side view showing an example of a surgical microscope system 60, and FIG. 1(b) is a detailed perspective view showing an end portion of the surgical microscope system 60.

At the end portion, the surgical microscope system 60 has a surgical microscope 61 and a pair of binoculars 62. Optical image data acquired by the surgical microscope 61 are electronically processed and transmitted to the binoculars 62, where images are displayed on a display panel. Thus, two peoples including a doctor, a nurse and the like can observe the acquired images by the surgical microscope system 60 simultaneously. As shown in FIGS. 1(a) and 1(b), the surgical microscope system 60 comprises a base 67 and arms 66a, 66b and 66c, which are rotatably connected by joints 65a, 65b, 65c and 65d. The surgical microscope 61 is supported by a supporting arm 63 and the binoculars 62 are supported by a holding arm 64 independently from the surgical microscope 61. The binoculars 62 are rotatably fitted around an axis. As shown in FIG. 1(b), the surgical microscope 61 has an objective lens 61a and eyeglasses 61b, and the binoculars 62 have eyeglasses 62a.

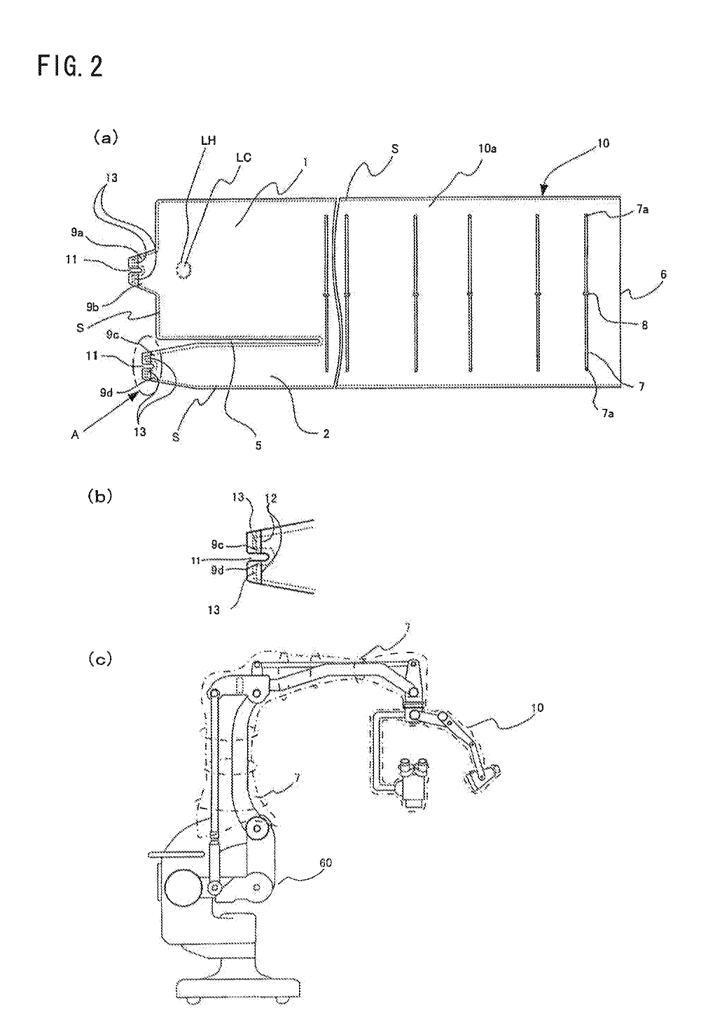

FIG. 2(a) shows the first embodiment of a microdrape 10. FIG. 2(c) shows a surgical microscope system 60 covered with microdrape 10.

As shown in FIG. 2(a), the microdrape 10 is a cylindrical bag having an opening end 6. The border of the microdrape 10 except the opening end 6 is joined by fusion or the like such that a joining hem S is formed. The microdrape 10 is formed from two transparent sheets made of polyethylene, polypropylene, polyvinyl chloride or the like with a length of at least ca. 4 m, ca. a width of ca. 1 m and a thickness of 100-300 μm. The inner side of the microdrape 10 is completely sterilized. Then the microdrape is folded into a small package and the small package is sterilized. Basically the microdrape 10 consists of a main body 10a having a cylindrical bag shape, a cylindrical bag 1 and a cylindrical bag 2. Middle parts of binding tapes 7 are fixed to the main body 10a by fixing tapes 8. The number of the binding tapes 7 is properly determined according to the size, height and the like of the surgical microscope system 60. The binding tapes 7 are employed in order not to restrict movement of the surgical microscope system as preventing the microdrape 10 from expanding. As shown in FIG. 2(c), after the surgical microscope system 60 is covered with the microdrape 10, the binding tapes 7 are wound around the covered surgical microscope system 60, so that the microdrape 10 is prevented from expanding. Double sided tapes 7a are attached on both ends of each binding tape 7 for fixing the binding tape 7 to the microdrape 10 after winding the binding tape 7 around the surgical microscope system 60.

In the side opposite to the open end 6, the microdrape 10 has the cylindrical bag 1 and the cylindrical bag 2. The bag 1 covers the surgical microscope 61 and the bag 2 covers the binoculars 62. Two projecting portions 9a, 9b for covering lens tubes of the eyeglasses 61b are arranged in the center of the end of the bag 1, such that the projecting portions 9a, 9b are facing each other across a slit 11. In the same way, two projecting portions 9c, 9d for covering lens tubes of the eyeglasses 62a are arranged in the center of the end of the bag 2, such that the projecting portions 9c, 9d are facing each other across a slit 11. Perforation lines 13 are formed on tip ends of the respective projecting portions. After the surgical microscope system 60 is covered with the microdrape 10, the tip ends are cut off from the projecting portions along the perforation lines 13. Further, rubber rings 12 are arranged near the perforation lines 13 for firmly fixing the projecting portions 9a, 9b, 9c and 9d to the lens tubes of the eyeglasses 61b and 62a after the lens tubes are covered with the respective projecting portions. FIG. 2(b) is the partially enlarged view of area A in FIG. 1(a). And a lens hood LH and a lens cover LA for covering the objective lens 61a are arranged near the projecting portions 9a, 9b. The lens cover LC is removed after the objective lens 61a is covered.

Both the bag 1 and the bag 2 are connected to the main body 10a, but they are separated by a slit 5. Due to the slit 5, a block consisting of the supporting arm 63 and the surgical microscope 61; and other block consisting of holding arm 64 and the binoculars 62 can be individually covered with the microdrape 10 quickly. The microdrape 10 is not damaged, even if, for example, the binoculars 62 are rotated together with the holding arm 64.

At first the tip area of the surgical microscope system 60 is inserted in the microdrape 10 as widening the open end 6 and finally the arms 66a, 66b are inserted in the microdrape 10. Then the lens tubes of the eyeglasses 61b of the surgical microscope 61 are inserted in the projecting portions 9a, 9b, and the projecting portions 9a, 9b are fixed to the lens tubes with the rubber rings 12. The lens tubes of the eyeglasses 62a of the binoculars 62 are inserted in the projecting portions 9c, 9d, and the projecting portions 9c, 9d are fixed to the lens tubes by the rubber rings 12. Then the objective lens 61a of the surgical microscope 61 is inserted in the lens hood LH and the lens cover LC is removed. The tip ends are cut off from the projecting portions 9a, 9b, 9c, 9d along the perforation lines 13.

When the surgical microscope system 60 is completely covered with the microdrape 10, the bag shaped main body 10a is firmly fixed to the surgical microscope system 60 by winding the binding tapes 7 around the covered surgical microscope system 60 so that the microdrape 10 is prevented from expanding.

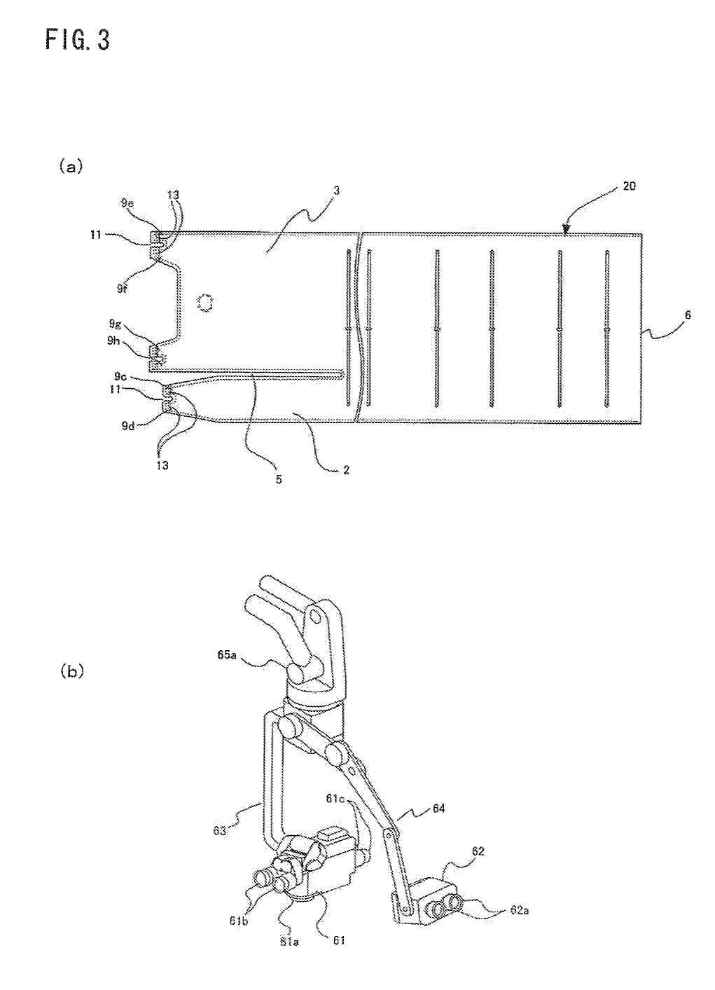

FIG. 3 shows the second embodiment. (a) is the plan view of a microdrape 20 and (b) is the partial perspective view of the surgical microscope system 60 to be covered with the microdrape 20 shown in (a). In the present embodiment, the surgical microscope 60 is equipped with two pairs of the eyeglasses 61b, 61c, so that a cylindrical bag 3 of the microdrape 20 has four projecting portions 9e, 9f, 9g, 9h. A cylindrical bag 2 of the microdrape 20 is the same as the cylindrical bag 2 of the microdrape 10 in the first embodiment. A slit 5 is formed between the cylindrical bag 2 and the cylindrical bag 3 in the same way as the microdrape 10 of embodiment 1. Since other arrangements are the same as the microdrape 10 of the previous embodiment, further detailed explanations are omitted.

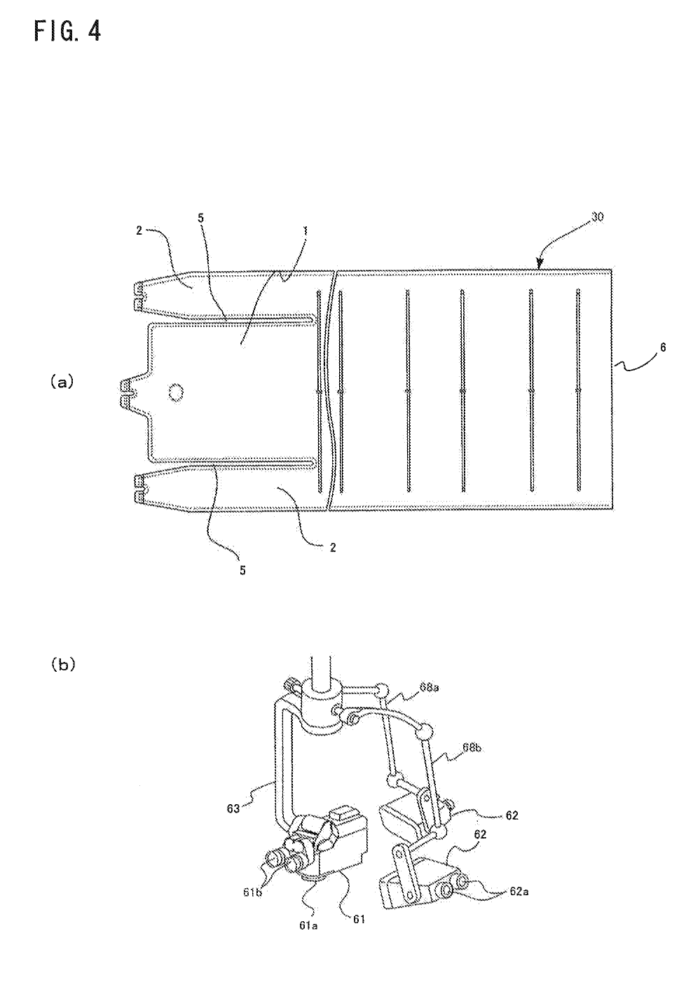

FIG. 4 shows the third embodiment. (a) is the plan view of a microdrape 30 and (b) is the partial perspective view of the surgical microscope system 60 to be covered with the microdrape 30 shown in (a). In the present embodiment, the surgical microscope 61 is equipped with a pair of the eyeglasses 61b and two pairs of binoculars 62 respectively supported by holding arms 68a, 68b independently. Due to this arrangement, the microdrape 30 has one cylindrical bag 1 for the surgical microscope 61 and two bags 2 for two pairs of binoculars 62, the bags having two projecting portions respectively. Two slits 5 are formed between the bag 1 and respective bags 2. In this way, even if the number of the binoculars is increased, bags for the increased binoculars can be formed without difficulties by forming the slit 5 between the two bags, so that the surgical microscope system can be covered with the microdrape rapidly and firmly. Since other arrangements are the same as the microdrape 10 of the first embodiment, further detailed explanations are omitted.

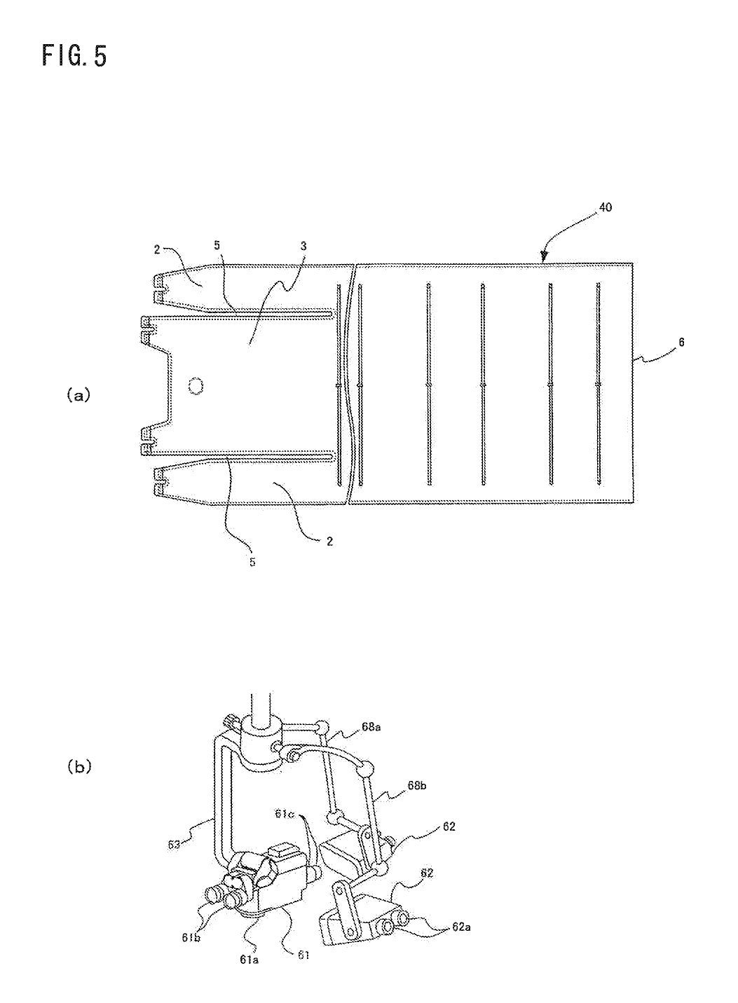

FIG. 5 shows the fourth embodiment. (a) is the plan view of a microdrape 40 and (b) is the partial perspective view of the surgical microscope system 60 to be covered with the microdrape 40 shown in (a). In the present embodiment, the surgical microscope 61 is equipped with two pairs of the eyeglasses 61b, 61c and two pair of binoculars 62. In this way, even if the number of the eyeglasses is increased, bags for the increased eyeglasses can be formed without difficulties, so that the surgical microscope system can be covered with the microdrape rapidly and firmly. Since other arrangements are the same as the microdrape 10 of the first embodiment, further detailed explanations are omitted.

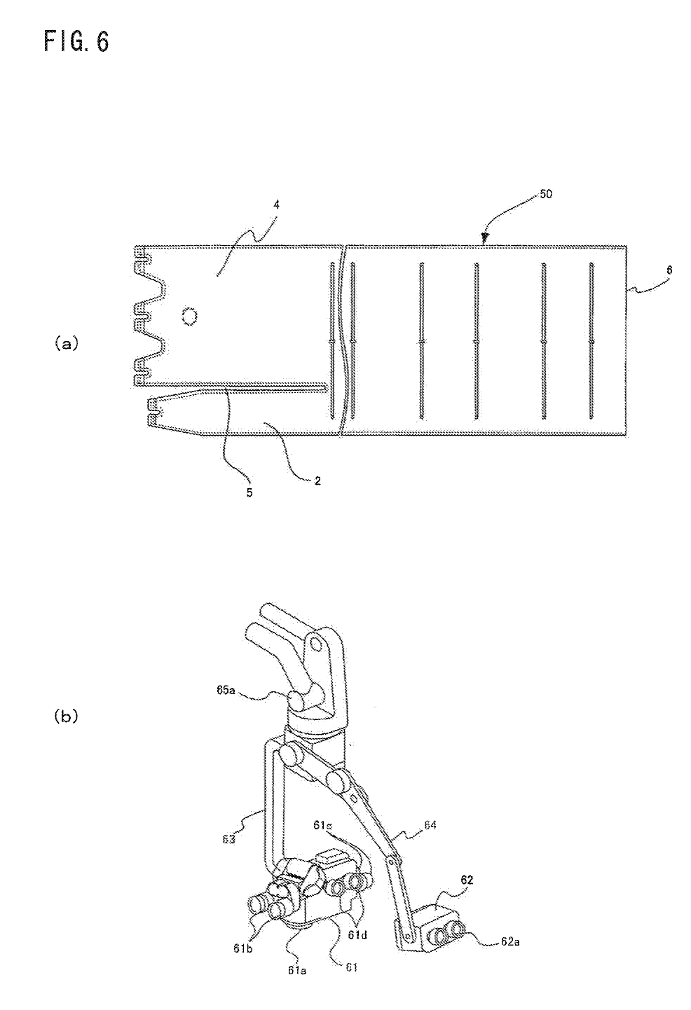

FIG. 6 shows the fifth embodiment. (a) is the plan view of a microdrape 50 and (b) is the partial perspective view of the surgical microscope system 60 to be covered with the microdrape 50 shown in (a). In the present embodiment, the surgical microscope 61 is equipped with three pairs of the eyeglasses 61b, 61c, 61d and a pair of binoculars 62. Due to this arrangement, the microdrape 50 has a bag 4 with 6 projecting portions for three pairs of eyeglasses. In the same way as in the previous embodiments, a slit 5 is formed between the bag 4 and the bag 2. Since other arrangements are the same as the microdrape 10 of the first embodiment, further detailed explanations are omitted.

As explained above, embodiments of the present invention provide microdrapes, which can properly cover a surgical microscope, binoculars, arms for supporting the binoculars and the like without disturbing the movements of the arms.

### REFERENCE NUMERALS AND CHARACTERS

1 Bag (for a surgical microscope)
2 Bag (for binoculars)
5 Slit
6 Opening end
7 Binding tape
7a Double sided tape
8 Fixing tape
9a, 9b, 9c, 9d Projecting portions (for inserting lens tubes of eyeglasses)
10 Microdrape
10a Main body of the microdrape
11 Slit
12 Rubber ring
13 Perforation line
60 Surgical microscope system
61 Surgical microscope
62 Binoculars (for assistant)

What is claimed is:

1. A microdrape for covering a surgical microscope system comprising a surgical microscope and one or more pair(s) of binoculars independently supported by one or more arm structure(s), wherein the microdrape has an opening end and a closed end opposite to the opening end, the microdrape comprising:
a cylindrical bag shaped main body having said opening end;
a first cylindrical bag connected to said main body, wherein the first cylindrical bag has a hood for covering a lens tube of an objective lens of said surgical microscope and projecting portions for covering lens tubes of eyeglasses of said surgical microscope; and
one or more second cylindrical bag(s) connected to said main body, wherein the second cylindrical bag(s) has projecting portions for covering lens tubes of eyeglasses of said one or more pairs of binoculars, wherein:
one or more slit(s) are formed between said first cylindrical bag and said second cylindrical bag(s).

2. The microdrape according to claim 1, wherein:
said surgical microscope has a plurality of eyeglasses, and
said first cylindrical bag has a plurality of projecting portions for covering lens tubes of said plurality of eyeglasses of said surgical microscope.

3. The microdrape according to claim 1, wherein:
perforation lines are formed on tip ends of the projecting portions for covering the lens tubes of the eyeglasses of said surgical microscope and on tip ends of the projecting portions for covering the lens tubes of eyeglasses of said binoculars, and
rubber rings are arranged near said perforation lines.

4. The microdrape according to claim 1, wherein:
a predetermined number of binding tapes are arranged on said cylindrical bag shaped main body a predetermined space apart.
